(19) United States
(12) Patent Application Publication
(10) Pub. No.: US 2010/0042225 A1
(43) Pub. Date: Feb. 18, 2010

(54) KNEE PROSTHESIS

(76) Inventor: Vladimir Shur, Short Hill, NJ (US)

Correspondence Address: Joel D. Citron, 2003 Ferndale Drive, Wilmington, DE, 19810, US

(21) Appl. No.: 12/449,838

(22) PCT Filed: Aug. 22, 2008
(86) PCT No.: PCT/US2008/009980

§ 371 Date: Aug. 31, 2009

Related U.S. Application Data
(60) Provisional application No. 61/065,415, filed on Feb. 11, 2008.
(60) Provisional application No. 60/968,157, filed on Aug. 27, 2007.

(51) Int. Cl. A61F 2/38

(52) U.S. Cl. 623/20.35; 623/20.14

(57) ABSTRACT

A femoral component for a knee prosthesis comprises a reduced wear polyethylene (RWPE). Preferably all of the femoral component is the RWPE and/or at least one condylar surface of the femoral component is the RWPE. The use of RWPE instead of conventional metal components reduces or eliminates complications associated with the use of metal such as fracture (mostly at the area of the bone-metal interface), mechanical failure, and metallosis. Use of the RWPE gives a prosthesis with long life, especially with respect to wear of the femoral component.

### FIELD OF THE INVENTION

The present invention relates to an improved knee prosthesis for use within the body, wherein the femoral component includes reduced wear polyethylene.

### BACKGROUND OF THE INVENTION

Prosthetic implants are well known medical replacements for articulating parts of the human body, such as the hip, finger, spine, elbow, ankle, knee and the like. For example, a typical human knee joint includes a tibial, femoral and other known components. The knee joint components articulate in response to forces that are initiated during normal activities, such as walking, stepping, running or jumping. During articulation of the knee joint, the flexion and extension of the distal end of the femur (known as the “femoral condyles”) and the proximal end of the tibia (known as the “tibial plateau”) occurs about a transverse axis, with some degree of medial and lateral rotation along a longitudinal axis. The flexion, extension and rotation of the components of the knee joint allows movement so that an individual can carry out activities. Lateral and medial collateral ligaments, along with the menisci and muscles that transverse the joint, assist in controlling the movement of the knee's intended range of motion. For a knee, flexion is about 120° when the hip is extended, approximately about 140° when the hip is flexed, and about 160° when the knee is flexed passively. Medial rotation is limited to about 10° and lateral rotation is limited to approximately about 30°. During use, the knee joint will experience different ranges of motion, depending upon the activity.

Total knee or partial (such as unicompartmental) replacement and repair or replacement of existing prostheses is well known as one of the available surgical procedures to replace a damaged knee joint or prosthesis. During a total or partial knee replacement, an incision is made by a surgeon in a knee portion of a leg of a patient, using known procedures. The patella (knee cap) is everted from its normal position and the ends of the femur and tibia are shaved to eliminate any rough areas and to allow a prosthesis to be positioned into the knee joint. The procedures used for a total or partial knee replacement are known, and have been described by a number of patents, such as for example U.S. Pat. No. 7,104,996 to Bonutti, U.S. Pat. No. 7,081,137 to Servido, U.S. Pat. No. 6,859,661 to Tuke, U.S. Pat. No. 4,952,213 to Bowman et al., U.S. Pat. No. 4,470,158 to Pappas et al., U.S. Pat. No. 4,340,978 to Buechel et al., and U.S. Pat. No. 4,193,140 to Treace, each of which are incorporated herein by reference.

Prior art knee prosthesis utilizes a femoral component having highly polished and strong metal condyles as part of a metallic femoral component to provide maximum coverage of the distal femur. The condyles, which are typically made of cobalt, chrome or titanium, are configured to articulate against a bearing component that is affixed to the proximal end of the tibia. The tibial component supports the bearing component that is commonly made from polyethylene (PE), such as ultra high molecular weight polyethylene (UHMWPE). The UHMWPE is used to reduce friction and allow the metallic femoral component to move freely as the knee joint is articulated. Depending upon the condition of the knee cap, a patella component, which is typically made with durable plastic, is also used. An example of this type of prosthesis is shown by U.S. Pat. No. 5,957,979 to Beckman et al., which is incorporated herein by reference.

The use of metal for the femoral and sometimes other components of a knee prosthesis present unique challenges. Metal is a much stiffer material than human bone. The insertion of the metal into the femur can cause a series of well recognized complications, such as fracture (mostly at the area of the bone-metal interface), mechanical failure, and metallosis, to name several examples. Metal can also be difficult to remove without removing large quantities of a patient's bone, which makes repeated (revision) surgery more time consuming and complex. It is known that when a prosthesis must be removed and a revision prosthesis inserted, it is not uncommon for additional bone to be removed in order to stabilize the new prosthesis. During revision surgery, interior portions of the femoral component of the prosthesis is often augmented to compensate for the bone material that has been removed. As a result, the bone in the area of the revision surgery may become weaker due to repeated surgery at that location.

In opposition to metal, plastic is more similar to human bone characteristics and biomechanical parameters and has been used for different components of a prosthesis. For example, U.S. Pat. No. 6,464,926 to Merrill et al. discloses a process of making UHMWPE medical prosthesis for use within the body. The UHMWPE, as discussed therein, has a polymeric structure which is less than about 50% crystallinity, reduced lamellar thickness and less than about 940 MPa tensile elastic modules, to reduce the production of fine particles from the prosthesis during wear of the prosthesis. Merrill teaches that the UHMWPE based prosthesis disclosed in the '926 Patent is useful for contact with metal containing parts formed of, for example, stainless steel, titanium alloy, or nickel cobalt alloy. The process shown in Merrill contemplates a prosthesis device formed, in part, by a combination of metal and the UHMWPE disclosed therein. Merrill does not teach use of a prosthesis made of plastic for substantially all components or a UHMWPE that has reduced wear and maintains desired yield and tensile strength, in order to prolong the life of the particular component.

The use of plastic as a replacement for certain components of a prosthetic knee was impossible in the past, because of wear and poor durability of known orthopedic polymer materials. For instance, adhesive wear of tibial and patella components occurs under load and motion due to the interaction between the contacting surfaces. The motion of the components, under load, produces PE particles that can become lodged between contacting or load bearing surfaces. Abrasive wear occurs if the femoral component is roughened or scratched by the small particles, but can also occur from third bodies such as bone cement interposed between the bearing surfaces. Wear is not limited to the bearing surfaces but can occur at the back surface of modular components, e.g. between the PE tibial insert and the metal tray. Particles are small and can be liberated in large quantities. Small particles are known to elicit a higher tissue reaction and can result in osteolysis.

High contact stresses at the knee due to the non-conforming geometry of the components results in other wear characteristics at the knee. These characteristics include pitting and delamination. Pitting occurs due to the removal of small localized amounts of material. This phenomenon does not result in high amounts of wear but is indicative of high cyclic contact stresses that may lead to more significant wear such as delamination. The delamination phenomenon is accompanied by removal of sheets of material and is the end result of subsurface cracks that propagates below the surface and finally to the surface. Large amounts of material may be liberated by delamination. Although, the wear particles resulting from delamination are large, entrapment of the material between the bearing surfaces can result in the production of much smaller particles that can elicit a biological response.

Wear in total knee prosthesis is influenced by knee design, contact stress and kinematics, by component orientation and soft tissue structures, and of course materials of construction. To minimize wear, the design of knee components must use of material(s) that has (have) the desired mechanical properties to withstand the loads and movement stresses associated with knee movement. It should be understood that mechanical and fatigue properties of UHMWPE, if used, must be maintained in order to minimize pitting and delamination wear. Contact stress reduction via increased contact area will also reduce the incidence of pitting and delamination.

Prior art knee prosthetic devices have not incorporated UHMWPE based materials in femoral components, particularly, crosslinked, low wear UHMWPE material. Accordingly, it is desired to provide a prosthetic knee having a femoral component comprising reduced wear ultrahigh molecular weight polyethylene (RWPE).

It is also desired to provide knee prosthesis having a femoral component comprising RWPE, that overcomes the threat of short and long-term complications during and after surgery, and that are more biologically sound to the human body.

It is also desired to provide a full or partial medical prosthesis for use within the body, for a knee, that has improved performance capabilities.

U.S. Pat. No. 4,034,418 describes knee prostheses in which the femoral component comprises and high density polyethylene, while U.S. Pat. No. 5,358,529 describes a knee prosthesis in which the femoral component comprises ultra high molecular weight polyethylene. In neither reference is the use of a reduced wear polyethylene suggested, and other features described below are also not present.

D. J. Moore, et al., The Journal of Arthroplasty, vol. 13, (4), 1998, p. 388-395, “Can a Total Knee Replacement Prosthesis be made Entirely of Polymers?” describe a knee prosthesis in which the femoral component is made of polyacetal (Delrin®). A prosthesis in which a femoral component comprising reduced wear polyethylene is not mentioned.

### SUMMARY OF THE INVENTION

The present invention relates to a femoral component for a knee prosthesis, wherein said femoral component comprises a reduced wear polyethylene.

Also described herein is a full or partial knee prosthesis comprising the above described femoral component, and a process for replacing or partially replacing a knee or knee prosthesis using a full or partial knee prosthesis comprising the above described femoral component.

### BRIEF DESCRIPTION OF THE DRAWINGS

FIG. 1 is a front plan view of a portion of a human knee joint with a prior art prostheses, the knee being illustrated in an extension position.

FIG. 2 is a front plan view of a portion of a human knee joint with a medical prosthesis of the present invention, the knee being illustrated in an extension position.

FIG. 3 is a side plan view of the human knee joint and prosthesis shown in FIG. 2, illustrating the location of the patella components of the knee.

FIG. 4 is an isolated exploded perspective view of the medial prosthesis of the present invention, showing a femoral component, a load bearing spacer component, and a tibial component.

FIG. 5 is an isolated side view of a cross-section of the femoral component of the medical prosthesis shown in FIG. 4, taken along line 5-5

FIG. 6 is an isolated side view of a cross-section of load bearing component of the medical prosthesis shown in FIG. 4, taken along line 6-6.

FIG. 7 is an isolated side view of a cross-section of the tibial component of the medical prosthesis shown in FIG. 4, taken along line 7-7.

FIG. 8 is an isolated perspective view of an alternative embodiment of a femoral component of the present invention.

### DETAILED DESCRIPTION OF THE INVENTION

Turning now to the drawings, wherein like numbers present like elements, there is shown embodiments of the present invention that are presently preferred. The present invention is directed to a knee prosthesis having improved components to be used within a human or animal body. It is contemplated that the components of the knee prostheses of the present invention may be made of the same or different materials; can be shaped, size and dimensioned relative to the size of the user or the type of activities to be engaged in by the user; and/or made of one or more materials that are predetermined by a surgeon based upon the interests of a given patient, such interests including reactions or resistance to the use of certain materials by patients, reduction in the likely need for revision surgery, reduction in the risk of fractures in the bone or material of the components, and a reduction of wear rates, as several examples.

### A. Prior Art Prosthesis

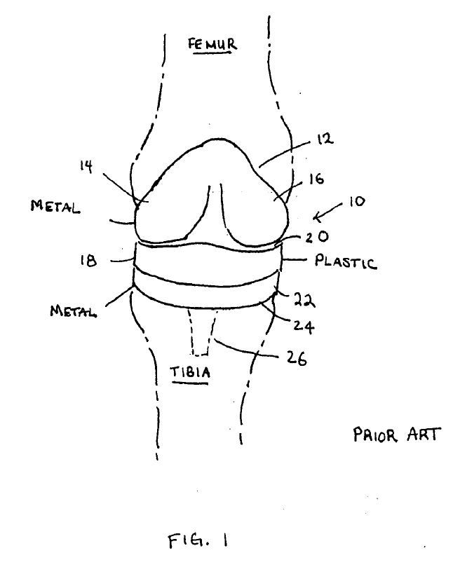

FIG. 1 shows a prior art prosthesis for a knee joint 10, which is commonly used for a total or partial knee replacement of a knee or in revision surgery. The knee prosthesis 10 comprises a femoral component 12 attached to the distal end of a femur (shown in phantom), having a condylar surface 14 and 16 that are shaped to slideably engage a spacer 18. The femoral components are made of durable, non-coercive highly polished metallic material, such as chrome, titanium alloy or platinum. The metallic material of the femoral component is used to provide a relatively smooth, but durable surface so that the condylar surfaces (these surfaces are “wear surfaces”, that is they experience wear by moving against an articular surface of 20) 14 and 16 will freely rotate on an articular surface 20 of the spacer 18.

Spacer 18 is typically made of UHMWPE and has generally concave, spherically shaped recesses (not shown) formed about the articular surface 20 that substantially correspond to the shape of the condylar surfaces 14 and 16. The spacer 18 is mounted to a tibial component 22 that is secured to the proximal end of the tibia (shown in phantom). The tibial component 22 has a tray or mounting platform 24 and a mounting post 26 (shown in hidden lines) that extends away from the mounting platform 24 that is shaped and dimensioned to be fixedly, but releasably secured to the spacer 18. The spacer 18 and tibial component 22 are secured to the tibial using known techniques and mounting materials that are known in the art, such as adhesives and bone cement.

As shown, the prior art knee prosthesis 10 used in the art utilizes a combination of metallic components with a layer of PE material at the interface between the condylar surfaces 14 and 16 and the articular surfaces 20. Metal has been the preferred material used for at least the femoral and tibial components 12 and 22, respectively, due to the physical characteristics of metallic material, such as titanium, although plastic has been used at times for the tibial component. Use of highly durable metallic material provides a much stiffer material than human bone and, in the case of the femoral component 12, enable the manufacture to create a highly polished smooth surface. However, the use of metal can increase the risk of complications, such as fracture of the bone due to the metal-bone interface (mostly about the bone-metal junction), mechanical failure of the bone, metallosis, and the like. In addition, the combination of the use of a metallic femoral component and a PE spacer can causes problems for the user. For example, the metal of the femoral component is known to cause degradation of the physical properties of the plastic, due to repeated loading and unloading and sheer stress that arise when the prosthesis is under use. The forces (traverse, axial and rotational) that are imparted to the knee portion of a human being or animal, cause significant wear of the components that are used to make the components of the prosthesis. As a result of that wear, small particles of the plastic material can develop about the metal-to-plastic interface that can get logged between the condylar surface and the spacer 18 which, in effect, can impede the performance and operation of the prosthetic knee.

Many other variations of knee prostheses are known, see for instance U.S. Pat. Nos. 7,104,996, 7,081,137, 6,859,661, 4,952,213, 470,158, 4,340,978, and 4,193,140.

The present invention overcomes the problems associated with using metal femoral components of a prosthetic knee joint by providing a prosthesis that has the type of mechanical and biological stability, and wear resistance, that will prolong the useful life of the prosthetic joint.

### B. A Preferred Embodiment of the Present Invention

The present invention is often similar to prior art prostheses except the femoral component comprises an RWPE.

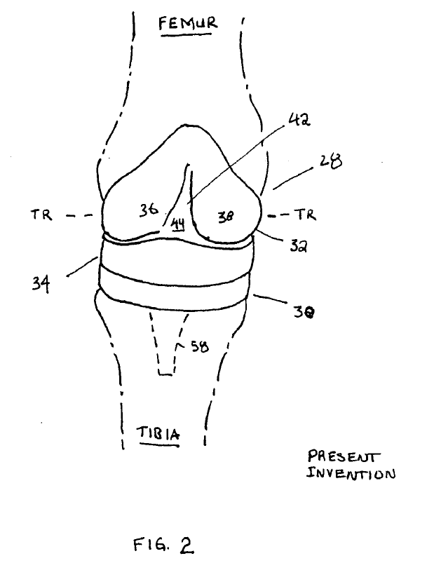

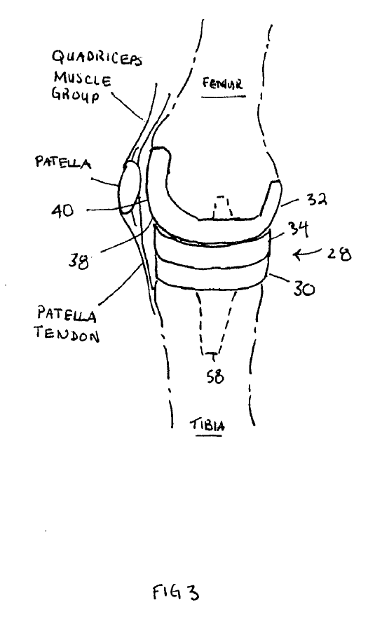

FIG. 2 shows a modular medical prosthesis 28 as contemplated by the present invention. As shown, the prosthesis 28 comprises a femoral component 32 secured to the distal end of an exemplary femur (shown in phantom), a load bearing spacer component 34 and a tibial tray component 30 secured to the proximal end of an exemplary tibia (also shown in phantom). As shown, in FIG. 3, the front portion of the femur and tibial that face the left side of the paper will be seated close to and behind a patella that is joined at one end to the quadriceps muscle group and the other end to the patella tendon. The modular structure of the prosthesis 28, particularly the spacer component 34, of the present invention is advantageously used as a means to reduce, if not avoid, significant or complete interference with the bone and/or components of the knee joint that remain after the prosthesis is inserted.

Returning to FIG. 2 or 3, the femoral component 32 is fixedly, but removably secured to the distal end of the femur using known securing means, such as by friction, adhesives, bone cement and the like. The femoral component 32 has one large or a pair of condylar portions 36 and 38 that form a substantially curved condyle surface 40, as also seen in FIG. 3. Condylar portions 36 and 38 are seamlessly joined about a region 42 (FIGS. 2 and 4) positioned anterior of the condyle surface 40 to form a recess or channel region 44. Region 44 runs approximately centrally about the symmetrical axis 46 of the femoral component 32, as best seen in FIG. 4. Continuing with FIG. 4, the side of the femoral component 32 that faces the femur preferably includes, as an option, a pair of spaced apart mounting pins or post 50 and 52, that function analogous to the prior art. Pin 50, as well as other components of the femoral component 32, are a mirror image of pin 52. Further discussion of the structure of the mounting pins 50 and 52 is not believed necessary because their function is understood in the art. It is contemplated that the femoral component can be made with or without mounting pins 50 and 52, as best seen in FIG. 8 and identified by reference number 32′.

Returning to FIG. 2, the tibial component 30 comprises a tray or support member 54. As best seen in FIG. 4, the tray 54 is defined by a planar support member or plate 56 that is joined at its first and second (not shown) sides by a tapered keel or spike 58 that depends away from the second side of the plate 56. The plate 56 is substantially planar so that it will provide a mounting surface for the load bearing insert 34, as shown in FIG. 4. The load bearing insert or spacer 34, is mounted within the prostheses 28 intermediate the femoral component 32 and the tibial component 30. The spacer 34, which in an alternative embodiment may be integrally formed with or into the tibial component 30, includes a superior load bearing surface 61, defined in part by a pair of articular surfaces 60 and 62, that are preferably, but not necessarily, mirror images of each other. The articular surfaces 60 and 62 that are slightly depressed below the surface 61 to form load bearing areas that correspond in a one-to-one mating relationship to with condylar surfaces 36 and 38. The articular surfaces enable the space 34 to slideably engage the condylar surfaces 36 and 38 of the femoral component 32, thus allowing movement of the knee joint. When being used, the interface and interaction between the condylar surfaces 36 and 38 and the articular surfaces 60 and 62 provided a means in which the femoral component 32 can rotate or pivot about a transverse axis of rotation “TR”, as best seen in FIG. 2.

In practice, the femoral component 32 will rotate with respect to the tibial component about TR as the person flexes and extends the knee during activities, such as walking, sitting, running, bounding stairs, exercising, and the like. It should be understood that the condylar surfaces 36 and 38 are shaped and dimensioned to correspond to the articular surfaces 60 and 62 so that the pivoting or rotation of the femoral component can achieve a desired range of motion. It should also be understood that there is a degree of sliding motion that occurs with normal use of the prosthetic knee that occurs when used. The slideability of the components of the prosthetic knee 28 is achieved by use of the interface between the condylar surfaces 36 and 38 and the articular surfaces 60 and 62 about the spacer 34.

Spacer 34 preferably includes a protrusion or post 64 that is preferably located intermediate the articular surfaces 60 and 62 (FIG. 4). The post 64 projects away from the load bearing surfaces 60 and 62 to engage the recess 48 of the femoral component. The post 64 is provided to guide the movement of the femoral component 32 relative to the spacer 32 or tibial component 34 and to prevent hyperextension of a person's knee beyond a desired or predetermined point. It is contemplated that the spacer 34 can be made with or without post 64, as illustrated in FIG. 8 and identified by reference numeral 34′.

Preferably, each of the components of the prostheses of the present invention are made of a material to reduce wear, increase the longevity of the prosthesis 28 and to maintain the mechanical strength and integrity of the knee joint during normal use over an extended period of time. It is contemplated that the spacer component 34 can be made of plastic (such as UHMWPE or RWPE), ceramic material or metal, which, overall, can be different than the materials used for the other components of the prosthesis 28. Due to the modular construction, the spacer can be replaced during replacement surgery without any significant or appreciable interference with the bone that is releasably attached to the remaining components, namely the femoral and tibial components. For example, the spacer component 34 can be made of a highly polished metal material (such as titanium), when the femoral and tibial components are made of a plastic (such as UHMWPE or RWPE, the latter especially for the femoral component)based material. Metallic material may be used for the spacer component 34 because it is easy to polish, will have low friction, and with reduce the wear of the plastic components, such as a RWPE based femoral component.

It is also contemplated that the prosthesis 28 may include a tray 54 that is made of plastic (such as RWPE or UHMWPE) or metal. The present invention allows flexibility in choosing the material used for the various components of the prosthesis 28 that is predetermined or pre-desired by the user, surgeon or manufacture. All of the material chosen should extend the useful life of the prosthesis 28, when used under normal circumstances by a person, which circumstances includes activities such as walking, running, squatting, lifting, driving, biking, walking on stairs, and the like, while at the same time do as little damage to the tissue (bones and soft tissue) in its vicinity, so that revision surgery, if needed, will be less traumatic.

The femoral component comprises a RWPE of the type described immediately below. It should be understood by those of ordinary skill in the art that the material used for the femoral component can be used for all components. Therefore, the description of the material described herein can be used for one, two or all of the components of the prosthesis 28. It is also contemplated that the components, other than the femoral component, of the prosthesis 28 can be made of different material, such as ceramic material, metal, plastic (such as UHMWPE) or a combination thereof.

In the femoral component and knee prosthesis (as appliccable) of the present invention, it is preferred that metal or ceramic which is present in the femoral component does not contact the femur when the femoral component is in place in the body, and/or that metal or ceramic which is present in the tibial component does not contact the tibia when the tibial component is in place in the body. For instance, the tibial component may comprise plastic, for example UHMWPE or RWPE that is in contact with the tibia when the tibial component is in place in the body. It is preferred that the superior load bearing surface 61 be metal, ceramic or plastic, especially metal or ceramic, particularly when one or both of the condylar surfaces 36 and 38 of the femoral component are RWPE. In other words it is preferred that RWPE condylar surfaces of the femoral component “wear” against a ceramic or metal surface. It is especially preferred in the knee prosthesis that metal or ceramic which is present in the femoral component does not contact the femur when the femoral component is in place in the body, that metal or ceramic which is present in the tibial component does not contact the tibia when the tibial component is in place in the body, the condylar surfaces of the femoral component wear against a metal or ceramic surface, particularly when the condylar surfaces of the femoral component are RWPE. The metal or ceramic is considered in contact with the tibia or femur if it is in direct contact or separated from the bone by merely a thin layer of adhesive or other material to improve adhesion to the bone and/or ingrowth of the bone into the metal.

It is further to be understood that any of the (preferred) conditions of the invention described herein may be combined with any number of other (preferred) conditions to describe another preferred state of the invention.

The femoral component 28 comprises a RWPE. The RWPE of the type contemplated for use with the prosthesis of the present invention, should provide a combination of physical characteristics that maintain desired mechanical and fatigue strength relative to the forces and stresses that are experienced by a prosthetic device, such as a prosthetic knee, under normal use by an individual. The RWPE should preferably be selected to have a bearing surface and mechanical integrity to withstand the anticipated activity of patients (such as walking, running, skiing, climbing, dancing, driving, lifting, pulling and the like), while maintaining stability.

RWPE is a crosslinked PE which is suitable for medical (prosthesis) use, and is made from a polyethylene which, for instance, meets the requirement of ASTM Specification F648-04. The PE before crosslinking is preferably an UHMWPE and has a weight average molecular weight of at least 3×10⁵, preferably at least about 5×10⁵, and very preferably at least about 10×10⁵ Daltons. The molecular weight may be measured by Size Exclusion Chromatography, using a PE standard calibration. UHMWPEs meeting ASTM F648-04 are commercially available, for example Ticona® GUR 1020 and GUR 1050 available from Celenese Corp., Dallas, Tex. 75234, USA.

The PE is crosslinked, usually by exposure to gamma radiation, in a controlled fashion (care must be taken not to degrade other polymer properties), to produce a RWPE, which is a crosslinked polymer. Such methods are known in the art, see for instance A, Wang, et al., Journal of Physics D: Applied Physics, vol. 39, p. 3213-3219 (2006), US Patent Applications 2005/0043431, 2007/0293647, 2007/0265369, 2007/0197679, and 2005/0010288, and U.S. Pat. Nos. 6,414,086, 6,095,511, 7,304,097 and 6,726,097, all of which are hereby included by reference.

In order to be a RWPE, the RWPE must have certain wear properties when compared to the uncrosslinked PE (UHMWPE for instance) from which it was made. The wear testing is done according ASTM Method F2025-06. Identical parts for a knee prosthesis are made from both the RWPE and the polyethylene (such as UHMWPE) from which it was made. These parts are then tested in a complete knee prosthesis according to ASTM F2025-06, section 4.2. After preparing the knee prosthesis specimens the wear tests are run using a simulator device which mimics human knee joint movements and loads, see for instance ISO 14243-2, as referenced in ASTM F2025-06. During the test the prosthesis should be lubricated with a suitable lubricant, as mentioned in the test method. The crosslinked PE which is being tested to determine if it is an RWPE and its uncrosslinked precursor shall be tested under conditions which are identical as possible. Although any part of the knee prosthesis made from these polymers may be tested, it is preferred that the femoral component, if it has RWPE wear surfaces [i.e. the condyle surface(s)] be tested. The other surface may be the same polymer or some other material such as metal or ceramic. If the femoral component containing the RWPE is meant for revision surgery and there is no corresponding wear surface being replace, it shall be tested against itself, that is uncrosslinked PE against uncrosslinked PE, and crosslinked PE against crosslinked PE. If one of the tested parts is a femoral component with RWPE wear surface(s), then the net volumetric wear of the femoral component shall be used to determine % RWPE_{w} (see below). To be an RWPE the crosslinked polymer must have 60% or less wear (% RWPE_{w}), preferably about 40% or less wear, and more preferably 20% or less wear than the uncrosslinked PE from which it was made.

The percent difference in wear (% RWPE_{w}) between the crosslinked (which is being tested to determine if it is an RWPE) and uncrosslinked PE is calculated using the equation:

% RWPEW=[(Vₙ of crosslinked PE)/(Vₙ of uncrosslinked PE)]100

Vₙ is the net volumetric wear (mm³) of each (crosslinked and uncrosslinked) PE sample as defined in ASTM Method F2025-06. The wear test is run for at least 1,000,000 cycles, preferably 5,000,000 cycles. An exemplary description of this method is found in A. Wang et al., J. Phys. D: Appl. Phys., vol. 39, p. 3213-3219 (2006), which is hereby included by reference, except that the femoral component in this reference is metallic. When testing whether a crosslinked PE is a RWPE the RWPE shall be tested against an opposing wear surface which is made from the same material against which it will wear in the actual prosthesis. If the condylar surface(s) of the femoral component are to be made of RWPE, they shall be wear tested against the material that will oppose them in the actual prosthesis, and the wear result for these taken as to whether the crosslinked PE is an RWPE. The opposing material for both the crosslinked and uncrosslinked PE condylar surface(s) shall be the same, whether crosslinked or uncrosslinked PE or some other material, and the same which is to be used in the actual prosthesis.

Preferably at least 50 volume percent of the femoral component is RWPE, more preferably at least about 75 volume percent, especially preferably at least about 90 volume percent, and very preferably all of the femoral component should be RWPE. Included within the meaning of RWPE are materials typically found in PEs such as antioxidants, crosslinking agents (and their decomposition products if any), fillers, reinforcing agents, and other small particle solids or other materials which are dispersed within the polymeric matrix.

The RWPE will often reduce scratches, wear striations, smearing and ripping of the load bearing and condylar surfaces at the junction between the femoral 30 component and the load bearing insert when compared to a similar component made from its uncrosslinked PE precursor.

RWPE of the type contemplated by the present invention is available from a number of manufacturers, such as: X3 from Stryker Orthopaedics; Advanced Polyethylene from Wright Medical Technology Inc., Arlington, Tex. 38002, USA; Prolong™ highly crosslinked PE from Zimmer, Inc.; ALTRX™ from DePuy Orthopaedics, Inc. (a subsidiary of Johnson & Johnson); and Arcom™ XL from Biomet, inc. The RVPE that is chosen can be selected based upon anticipated metal to plastic wear; plastic to plastic wear; the process in which the RWPE is made, and costs.

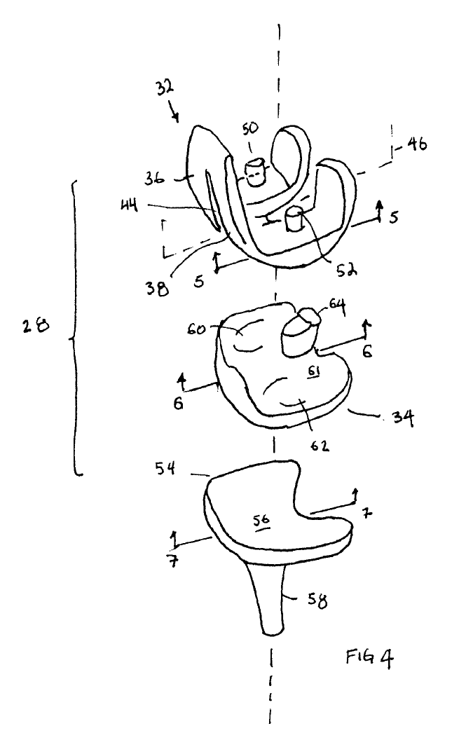

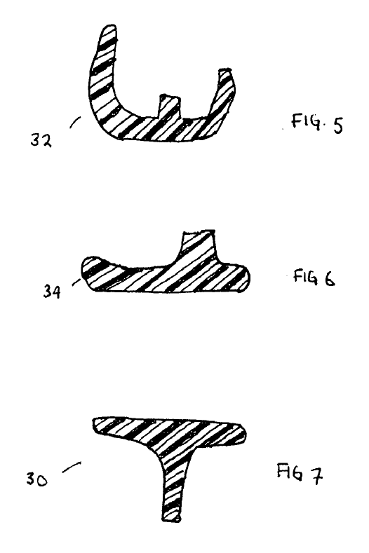

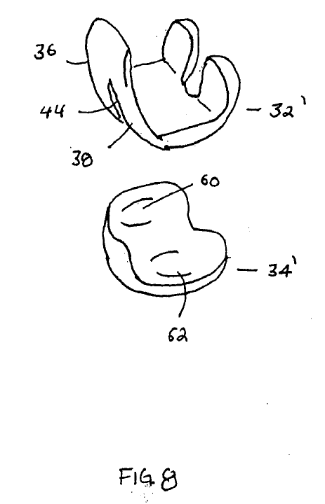

When using an RWPE several means can be used for mounting/inserting the components into the joint. For example, there are a number of bone cements available from a number of manufacturers, such as: Cobalt™ bone cement from Biomet, Inc., Warsaw, Ind. 46581, USA; DePuy®-1, -2, -3 and Smartset™ available from DePuy Orthopaedics, Warsaw, Ind. 46582, USA; Palacos™ Bone Cement available from Zimmer, Inc., Warsaw, Ind. 46581, USA; and Simplex™ P Bone Cement available from Stryker Orthopaedics, Mahwah, N.J. 07430, USA. If an adhesive or bone cement is used, it should be biologically suitable to a human body and not suffer a significant degree of degradation in its mechanical and/or adhesive characteristics, that can be present when adhesives are exposed to fluid and parts of the human body. FIGS. 5, 6 and 7, illustrate the use of a prosthetic knee 28 as contemplated by the present invention, in which all of the major components, namely the femoral component 32, the load bearing component 34, and the tibial component 30 are made of RWPE. The specific type of RWPE that is desired can be predetermined using known techniques in the art to simulate the wear and mechanical characteristics of the prosthetic knee when used. The life cycle or life of the prosthetic device, of the type described herein, can be lengthened by the selection of the type of RWPE in order to prevent revision surgery or other forms of surgery to replace components of the prosthetic device. Indeed, it is contemplated that the patella can be made of plastic, such as RWPE.

1. A femoral component for a knee prosthesis, wherein said femoral component comprises a reduced wear polyethylene.

2. The femoral component as recited in claim 1 wherein said femoral component is all reduced wear polyethylene.

3. (canceled)

4. The femoral component as recited in claim 1 wherein said reduced wear polyethylene is a crosslinked ultrahigh molecular weight polyethylene.

5. (canceled)

6. The femoral component as recited claim 1 wherein any metal or ceramic present in said femoral component does not contact a femur when said femoral component is in place in a body.

7. The femoral component as recited claim 4 wherein any metal or ceramic present in said femoral component does not contact a femur when said femoral component is in place in a body.

8. A knee prosthesis comprising the femoral component of claim 1.

9. The knee prosthesis as recited in claim 15 wherein any metal or ceramic in a tibial component does not contact a tibia when said tibial component is in place in said body.

10. The knee prosthesis as recited in claim 8 wherein a condylar surface of said femoral component wears against a metal or ceramic surface.

11. The knee prosthesis as recited in claim 9 wherein a condylar surface of said femoral component wears against a metal or ceramic surface.

12. A process for surgically completely or partially replacing a knee with an artificial prosthesis or repairing an artificial knee prosthesis, wherein the improvement comprises, using as a femoral component the femoral component of claim 1.

13. (canceled)

14. The knee prosthesis as recited in claim 8 wherein said reduced wear polyethylene is a crosslinked ultrahigh molecular weight polyethylene.

15. The knee prosthesis as recited in claim 8 wherein any metal or ceramic present in said femoral component does not contact a femur when said femoral component is in place in a body.

16. The knee prosthesis as recited in claim 15 wherein any metal or ceramic in a tibial component does not contact a tibia when said tibial component is in place in said body.

17. The process for surgically completely or partially replacing a knee with an artificial prosthesis or repairing an artificial knee prosthesis as recited in claim 12 wherein said reduced wear polyethylene is a crosslinked ultrahigh molecular weight polyethylene.

18. The process for surgically completely or partially replacing a knee with an artificial prosthesis or repairing an artificial knee prosthesis as recited in claim 12 wherein any metal or ceramic present in said femoral component does not contact a femur when said femoral component is in place in a body.

19. The femoral component as recited in claim 6 wherein said reduced wear polyethylene is a crosslinked ultrahigh molecular weight polyethylene.

20. The knee prosthesis as recited in claim 9 wherein said reduced wear polyethylene is a crosslinked ultrahigh molecular weight polyethylene.

21. The knee prosthesis as recited in claim 10 wherein said reduced wear polyethylene is a crosslinked ultrahigh molecular weight polyethylene.

22. The knee prosthesis as recited in claim 11 wherein said reduced wear polyethylene is a crosslinked ultrahigh molecular weight polyethylene.

23. The process for surgically completely or partially replacing a knee with an artificial prosthesis or repairing an artificial knee prosthesis as recited in claim 18 wherein any metal or ceramic in a tibial component does not contact a tibia when said tibial component is in place in said body.
